(19) United States
(12) Patent Application Publication
(10) Pub. No.: US 2019/0127345 A1
(43) Pub. Date: May 2, 2019

(54) NEGATIVE PHOTOCHROMIC MATERIALS WITH TUNABLE PROPERTIES

(71) Applicant: The Regents of the University of California, Oakland, CA (US)

(72) Inventors: Javier Read de Alaniz, Santa Barbara, CA (US); James Hemmer, Goleta, CA (US); Zachariah A. Page, Goleta, CA (US); Saemi Oh Poelma, Goleta, CA (US); Nicolas J. Treat, Santa Barbara, CA (US); Sameh Helmy, Goleta, CA (US)

(73) Assignee: The Regents of the University of California, Oakland, CA (US)

(21) Appl. No.: 16/096,063

(22) PCT Filed: Apr. 26, 2017
(86) PCT No.: PCT/US2017/029594

§ 371 Date: Oct. 24, 2018

Related U.S. Application Data
(60) Provisional application No. 62/327,683, filed on Apr. 26, 2016.

(51) Int. Cl. C07D 319/06; C07D 215/14; C07D 401/06; C07D 405/06; C07D 239/62; C07D 403/06; C09K 9/02

(52) U.S. Cl. CPC C07D 319/06; C07D 215/14; C07D 401/06; C09K 2211/1018; C07D 239/62; C07D 403/06; C09K 9/02; C07D 405/06

(57) ABSTRACT

Embodiments of the present disclosure describe a negative photochromatic material. Embodiments of the present disclosure further describe a method of tuning a negative photochromatic material comprising selecting an amine donor group, selecting an acceptor group, and contacting at least the selected amine donor group and the selected acceptor group to form a negative photochromatic material, wherein one or more of the selected amine donor group and the selected acceptor group tune at least an absorption range of the negative photochromatic material.

### BACKGROUND

Photochromic materials are a family of compounds which can undergo reversible photo-induced conversion between two different states or isomers with remarkably different properties. The study and synthesis of photochromic compounds has led to the development of a diverse array of “smart” materials, such as molecular logic gates, data recording and storage, nanoparticle delivery devices, rotary switches, sensors, and photo-controlled biological systems. Some of the most promising classes of photochromic materials are azobenzene, spiropyrans and diarylethenes. In general, photochromic compounds can be divided into two categories, T-type or P-type. T-type photochromes are those whose initial transformation is induced by light, while the back reaction is triggered by heat (ambient or elevated). In contrast, and perhaps the most studied, P-type photochromic compounds rely on two different wavelengths of light to regulate the conversion between states. One feature that is commonly shared between these privileged classes of photochromic compounds is that UV light is almost exclusively used to regulate the initial transformation. More recently, control has been achieved using longer-wavelengths of light (visible and NIR) by modifying the structure of photochromic material. This control is a major advance for the field because UV light is damaging to biological systems and can be detrimental to the stability of organic molecules.

Not surprisingly, there has been a number of efforts to design photochromic material that can be controlled using visible and near IR light. To this end, recent advances have been made in extending the wavelength of azobenzene photoswitches, however the synthesis which is poor yielding significantly hinders the potential use of these compounds. Photochromic material based on donor-acceptor Stenhouse adducts has also been disclosed. These derivatives switch from a conjugated, colored, and hydrophobic form to a ring-closed, colorless, and hydrophilic form on irradiation with visible light. Although the synthesis of these materials is more straightforward, the conversion between the different states only occurs in non-polar solvents, such as toluene, benzene, xylenes, and dioxane and the ability to tune wavelength is restricted. Further, reversible switching in polymeric systems could not be achieved. These limitations hinder the practicality of this system, as well as its use in material applications. On this basis, the ability to prepare photochromic material that provides tuneability for wavelength (i.e. visible to infrared), medium and switchability in polymeric systems would extend the potential utility of these compounds.

### SUMMARY

In general, embodiments of the present disclosure describe negative photochromatic materials and methods of tuning a negative photochromatic materials.

Accordingly, embodiments of the present disclosure describe a negative photochromic material characterized by the formula:

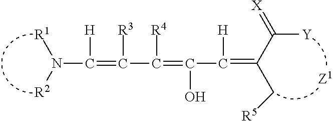

where R¹ and R² are independently one or more of alkyl, COOH, COO(C₁-C₂₀ alkyl), COO(aryl), aryl, heteroaryl, C₁-C₂₀ alkoxy, C₁-C₂₀ aryloxy, O(alkyl), O(aryl), S(alkyl) and S(aryl); wherein each alkyl and aryl group of R¹ and R² are independently one or more of alkyl, aryl, C₁-C₆ alkyl, heteroaryl, halogen, azide, hydroxyl, alkoxy, amino, N(C₁-C₂₀ alkyl), mono- or di-(C C₁-C₂₀) alkylamino, and halo(C₁-C₂₀) alkoxy; where R³ and R⁴ are independently one or more of hydrogen, alkyl groups, aryl groups, —S(aryl), —S(heteroaryl), —S(alkyl), —O(alkyl), —O(aryl), —O(heteroaryl), azide, and halogen; where R⁵ is independently one or more of —C═O, C═N-aryl, N═N-alkyl, C═NH, C═S, —CN, —CH═CH₂, CF₃, halo(C₁-C₂₀), alkyl, —COOH, —COO(C₁-C₂₀ alkyl), —COO(aryl), aryl, and heteroaryl; where X is independently O, N-aryl, N-alkyl, N—H, —(CN)₂, and S; where Y is independently one or more of O, N-alkyl, N-aryl, N-heteroaryl, S, alkyl, and aryl; and where Z¹ includes one or more of C, N, O, S, and Se. In some embodiments, each alkyl or aryl group of R¹ and R² may be optionally substituted with one or more groups that are independently one or more of alkyl, aryl, C₁-C₆ alkyl, heteroaryl, halogen, azide, hydroxyl, alkoxy, amino, N(C₁-C₂₀ alkyl), mono- or di-(C C₁-C₂₀) alkylamino, and halo(C₁-C₂₀) alkoxy. In addition, while Z¹ is described as including one or more of C, N, O, S, and Se, these shall not be limiting as Z¹ may include any nonmetallic atom that completes the cyclic ring.

Embodiments of the present disclosure further describe a method of tuning a negative photochromatic material comprising selecting an amine donor group characterized by the formula:

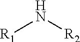

where R₁ and R₂ are independently one or more of alkyl, COOH, COO(C₁-C₂₀ alkyl), COO(aryl), aryl, heteroaryl, C₁-C₂₀ alkoxy, C₁-C₂₀ aryloxy, S(alkyl) and S(aryl); wherein each alkyl and aryl group of R¹ and R² are independently one or more of alkyl, aryl, C₁-C₆ alkyl, heteroaryl, halogen, azide, hydroxyl, alkoxy, amino, N(C₁-C₂₀ alkyl), mono- or di-(C C₁-C₂₀) alkylamino, and halo(C₁-C₂₀) alkoxy. The method further comprises selecting an acceptor group, and contacting the selected amine donor group and the selected acceptor group to form a negative photochromatic material, wherein one or more of the selected amine donor group and the selected acceptor group tune at least an absorption range of the negative photochromatic material.

### BRIEF DESCRIPTION OF DRAWINGS

This written disclosure describes illustrative embodiments that are non-limiting and non-exhaustive. Reference is made to illustrative embodiments that are depicted in the figures, in which:

FIG. 1 is a flowchart of a method of tuning a negative photochromatic material, according to one or more embodiments of the present disclosure.

FIG. 2 is a schematic view of DASA compounds synthesized with various donor components (boxed) which enabled tuning of wavelengths, according to one or more embodiments of the present disclosure.

FIG. 3 is a schematic view of aromatic amine based donor acceptor Stenhouse adducts, according to one or more embodiments of the present disclosure.

FIGS. 4A-B are graphical views of aromatic amine DASA absorption spectra, according to one or more embodiments of the present disclosure.

FIG. 5 is HOMO orbitals of aniline based DASAs, according to some embodiments.

FIG. 6 is a graphical view of NMR equilibrium open-closed, according to one or more embodiments of the present disclosure.

FIG. 7 is graphical views of cycling studies for an aromatic amine based DASA, according to one or more embodiments of the present disclosure.

FIG. 8 is a schematic view of solvent switching characteristics of dialkyl versus alkyl-aryl DASAs in solvents of varying polarity, according to one or more embodiments of the present disclosure.

FIGS. 9A-C are schematic and graphical views of a selective cyclization experiment, according to one or more embodiments of the present disclosure.

### DETAILED DESCRIPTION

The invention of the present disclosure relates to negative photochromatic materials. The negative photochromatic materials are highly tunable photoswitches. Photoswitches of the present disclosure are molecules that undergo a molecular change upon light irradiation. Upon excitation, the molecule transforms from its thermodynamically stable state to a photostationary state. The molecular change may include an isomerization that modifies the negative photochromatic material's absorption spectrum, polarity, molecular volume, or geometric configuration. These modifications to the negative photochromatic material, among others, may be used to control a range of properties, including, but not limited to, surface polarity, membrane permeability, surface patterning and nanoparticle clustering. For example, these negative photochromatic materials may be formed with highly tunable absorption wavelengths, as well as tunability with respect to media and switchability in both solution and polymeric systems. At least an amine donor group and an acceptor group may be selected to tune various properties of the negative photochromatic material. Embodiments of the present disclosure thus describe, among other things, these highly tunable negative photochromatic materials and methods of tuning these negative photochromatic materials to desired specifications.

The negative photochromatic materials of the present disclosure may be included in a highly tunable photoswitch system that can undergo a color change from a colored state to completely clear state, while maintaining synthetic routes and high tunability. One example of the present disclosure includes Donor Acceptor Stenhouse Adducts (DASA), which are a class of photoswitches that can be synthesized easily in two steps from commercially available starting materials. DASAs are considered negative photochromes, in that they reside in a conjugated colored form and, upon visible light irradiation, cyclize into a colorless form. DASAs absorb long wavelength visible light (545 or 570 nm), have high fatigue resistance, show a significant polarity change, and have a large volume change during the switching process. However, wavelength control via conventional methods has only been possible by altering the acceptor between Meldrums acid and barbituric acid. This limits the absorption profile of DASAs to two wavelengths, 545 nm and 570 nm, respectively. Though various secondary amine donors could be used, these had no effect on the wavelength.

Embodiments of the present disclosure describe a negative photochromatic material. In many embodiments, the negative photochromatic material is characterized by formula I:

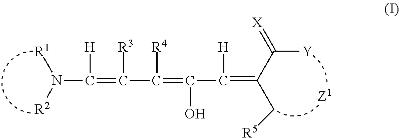

where R¹ and R² are independently one or more of alkyl, COOH, COO(C₁-C₂₀ alkyl), COO(aryl), aryl, heteroaryl, C₁-C₂₀ alkoxy, C₁-C₂₀ aryloxy, O(alkyl), O(aryl), S(alkyl) and S(aryl); wherein each alkyl and aryl group of R¹ and R² are independently one or more of alkyl, aryl, C₁-C₆ alkyl, heteroaryl, halogen, azide, hydroxyl, alkoxy, amino, N(C₁-C₂₀ alkyl), mono- or di-(C C₁-C₂₀) alkylamino, and halo(C₁-C₂₀) alkoxy; where R³ and R⁴ are independently one or more of hydrogen, alkyl groups, aryl groups, —S(aryl), —S(heteroaryl), —S(alkyl), —O(alkyl), —O(aryl), —O(heteroaryl), azide, and halogen; where R⁵ is independently one or more of —C═O, C═N-aryl, N═N-alkyl, C═NH, C═S, —CN, —CH═CH₂, CF₃, halo(C₁-C₂₀), alkyl, —COOH, —COO(C₁-C₂₀ alkyl), —COO(aryl), aryl, and heteroaryl; where X is independently O, N-aryl, N-alkyl, N—H, —(CN)₂, and S; where Y is independently one or more of O, N-alkyl, N-aryl, N-heteroaryl, S, alkyl, and aryl; and where Z¹ includes one or more of C, N, O, S, and Se. While Z¹ is described as including one or more of C, N, O, S, and Se, these shall not be limiting as Z¹ may include any nonmetallic atom that completes the cyclic ring. In some embodiments, the negative photochromatic material may generally be described as comprising one or more of an amine donor, a polymethine chain bearing oxygen heteroatom, and an acceptor group (e.g., electron acceptor group). The negative photochromatic material may be tuned by modifying, adjusting, and/or selecting one or more of R¹, R², R³, R⁴, R⁵, X, Y, and Z. In addition, the negative photochromatic material may be tuned according to any of the methods described herein.

In many embodiments, the negative photochromatic materials may be characterized as donor-acceptor Stenhouse adducts (DASA). In some embodiments, the negative photochromatic material may be characterized by one or more of the following chemical structures:

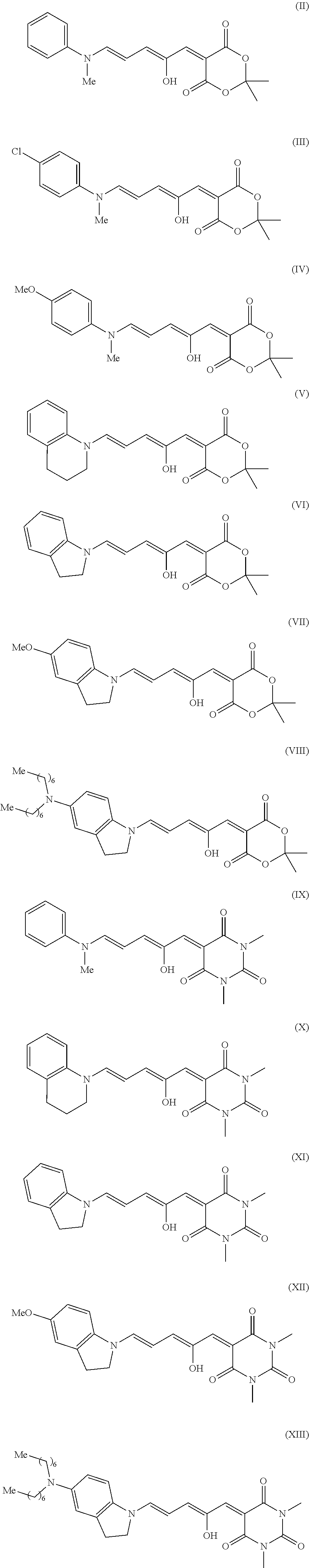

The negative photochromatic materials of the present disclosure may be tuned and/or modified to operate in a range of media according to any of the methods described herein. In many embodiments, the negative photochromatic materials may operate (e.g., complete or nearly complete photoswitching) in one or more of a polar medium, a non-polar medium, a solution phase medium, and a solid phase medium. The negative photochromatic materials may operate in a range of solutions (e.g., solvents) and/or in solid phase. For example, the negative photochromatic materials may operate in one or more of toluene, 1,4-dioxane, xylenes, anisole, dimethyl sulfoxide (DMSO), tetrahydrofuran (THF), water, methanol, ethanol, acetonitrile, chlorobenzene, N-methylpyrrolidone, dichlorobezene, trichlorobenzene, methylene chloride, acetone, benzene, cyclohexane, hexanes, ethyl acetate, diethyl either, 1,2-dichloroethane, and chloroform. In addition, the negative photochromatic materials may operate in a polymer matrix. The negative photochromic material can be absorbed into the polymer matrix, incorporated through post-functionalization or polymerized into the backbone of the polymer. Polymer compositions may include one or more (methy)acrylate, (meth)acrylamide, (meth)acrylonitrile, styrene, acrylonitrile, vinyl acetate, vinylcarbazole, vinylpyridine, vinyl ether, vinyl chloride, and siloxane monomers. Other solid phase medium may include, but are not limited to, paper, nylon, and/or fibers. In this case, the negative photochromic material can be absorbed into the medium and/or covalently attached through post-functionalization.

The negative photochromatic materials may also be tuned and/or modified according to any of the methods described herein to absorb wavelengths between about 400 nm and about 800 nm (e.g., about 530 nm to about 700 nm). Upon contacting the negative photochromatic material with electromagnetic radiation, the negative photochromatic material may convert from a thermodynamically stable state to a photostationary state. For example, the electromagnetic rotation can mediate an E to Z alkene isomerization that is followed by a thermally driven ring-closure to afford the colorless photostationary state. The conversion of the negative photochromatic material may induce a color change from a colored state to a completely colorless or nearly colorless state. The conversion of the negative photochromic material may also induce a polarity change from hydrophobic to hydrophilic upon contacting electromagnetic radiation. The conversion of the negative photochromatic material may also, or in the alternative, induce a molecular change (e.g., isomerization) that can be used to convert light into mechanical work. Combined or independently these property changes can be used to tune the negative photochromatic material's absorption spectrum, polarity, molecular volume, geometric configuration and/or control various properties, including, but not limited to, surface polarity, surface patterning, membrane permeability, and nanoparticle clustering.

A temperature dependence of the thermal reversion of the negative photochromatic materials may be tuned and/or modified according to any of the methods described herein. Temperature dependence can be tuned by modifying either the amine donor or acceptor group of the negative photochromic material which affects the switching kinetics of the system. In this case, the substituents can be used to modify either the sterics or electronics of the system to control the switching kinetics. Alternatively, the temperature dependence can be tuned by modifying the polymer glass transition (T_{g}). For example, going from a glassy to a rubbery matrix can be used to tune the kinetics of the thermal reversion with faster reversion being observed in rubbery matrix.

The negative photochromatic material and its tunable properties provide a material particularly suited for use in applications that include, among other things, photo-responsive drug delivery, photo-responsive phase-tag system, pigment, tattoo pigment, cosmetic pigment, data storage, re-writable systems, and sensors. This shall not be construed as limiting as the negative photochromatic materials may be used in a number of applications either known or unknown in the art.

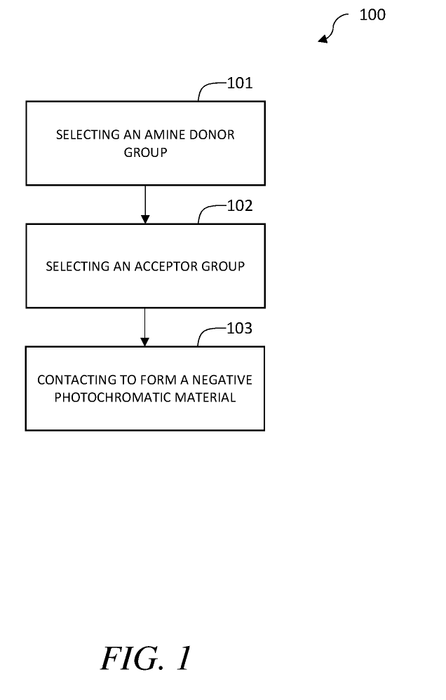

FIG. 1 is a flowchart of a method 100 of tuning a negative photochromatic material, according to one or more embodiments of the present disclosure. The method of tuning a negative photochromatic material comprises selecting 101 an amine donor group, selecting 102 an acceptor group, contacting 103 at least the selected amine donor group and the selected acceptor group to form a negative photochromatic material, wherein one or more of the selected amine donor group and the selected acceptor group tune at least an absorption range of the negative photochromatic material.

At step 101, an amine donor group is selected. In many embodiments, the selected amine donor group is a secondary amine and/or secondary aniline For example, the selected amine donor group may be characterized by the following formula:

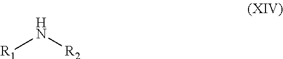

where R¹ and R² are independently one or more of alkyl, COOH, COO(C₁-C₂₀ alkyl), COO(aryl), aryl, heteroaryl, C₁-C₂₀ alkoxy, C₁-C₂₀ aryloxy, O(alkyl), O(aryl), S(alkyl) and S(aryl); wherein each alkyl and aryl group of R¹ and R² are independently one or more of alkyl, aryl, C₁-C₆ alkyl, heteroaryl, halogen, azide, hydroxyl, alkoxy, amino, N(C₁-C₂₀ alkyl), mono- or di-(C C₁-C₂₀) alkylamino, and halo(C₁-C₂₀) alkoxy. In some embodiments, the selected amine donor group is N-alkyl or N-aryl aniline, cyclic aromatic amines, cyclic amine heterocycles, and/or any other aromatic amine group. In some embodiments, R¹ and R² of the amine donor group may be selected such that the amine donor group is an N-alkylaniline, where alkyl can be C₁-C₂₀, wherein attached to the alkyl group is one or more of alkyl, aryl, C₁-C₆ alkyl, heteroaryl, halogen, azide, hydroxyl, alkoxy, amino, N(C₁-C₂₀ alkyl), mono- or di-(C C₁-C₂₀) alkylamino, and halo(C₁-C₂₀) alkoxy. For example, the amine donor group may include one or more of N-methylaniline, N-ethylaniline, N-propylaniline, N-butylaniline, N-pentylaniline, and N-hexylaniline.

The above embodiments relating to the selected amine donor group shall not be limiting as the selected amine donor group may include any secondary aromatic amine. Secondary aromatic amines play several important roles in the synthesis and the ability to control the properties of the negative photochromic material. In the case of synthesis, the aromatic amine may initially act as a nucleophile to ring open the activated furan ring that leads to the highly colored negative photochromic material. This requires that the aromatic amine be sufficiently nucleophilic to participate in the ring opening reaction and the groups (one or both) on the aromatic amine and nitrogen heteroatom can be used to tune/modify the nucleophilicity of the amine In the case of properties of the negative photochromic material, through careful choice of the amine donor group, the wavelength and solvent switching properties can be precisely controlled. For wavelength tunability the dihedral angle between the amine donor group and the conjugated triene is important, with diehdral angles of 0 being optimal. Also, to red-shift the absorption spectra an electron rich amine is optimal. For enhancing switching properties, the groups (one or both) on the aromatic amine and nitrogen heteroatom can be used to tune/modify the kinetics and equilibrium between the two states. At least these reasons, the amine group may include any secondary aromatic amine.

Accordingly, the amine donor group may include, for example, any aniline derivatives, tetrahydroquinoline derivatives, and/or indoline derivatives. For example, representative aniline derivatives may include one or more of N-methylaniline, N-ethylaniline, N-butylaniline, 4-bromo-N-methylaniline, and 4-cyano-N-methylaniline. In addition, representative tetrahydroquinoline derivatives may include one or more of tetrahydroquinoline, 6-methoxy-1,2,3,4-tetrahydroquinoline, and N,N-dimethyl-1,2,3,4-tetrahydroquinolin-6-amine Representative indoline derivatives may include one or more of indoline, indoline-2-carboxylic acid, 5-methoxyindoline, 5-methoxy-2-methylindoline, N,N-diethylindolin-5-amine, dihexylindolin-5-amine, N,N-diethyl-2-methylindolin-5-amine, and N,N-dihexyl-2-methylindolin-5-amine In preferred embodiments, the amine donor group includes one or more of N-methylaniline, N-ethylaniline, N-butylaniline, 4-bromo-N-methylaniline, 4-cyano-N-methylaniline, tetrahydroquinoline, indoline, indoline-2-carboxylic acid, 5-methoxyindoline, 5-methoxy-2-methylindoline, N,N-diethylindolin-5-amine, dihexylindolin-5-amine, N,N-diethyl-2-methylindolin-5-amine, N,N-dihexyl-2-methylindolin-5-amine, N-(2-(indolin-3-yl)ethyl)acetamide, N-(3-((4-methoxyphenyl)amino)propyl)acetamide, and 5-(diethylamino)indoline-2-carboxylic acid.

At step 102, the acceptor group is selected. In many embodiments, the selected acceptor group is a carbon acid acceptor group and may be characterized by one or more of the following:

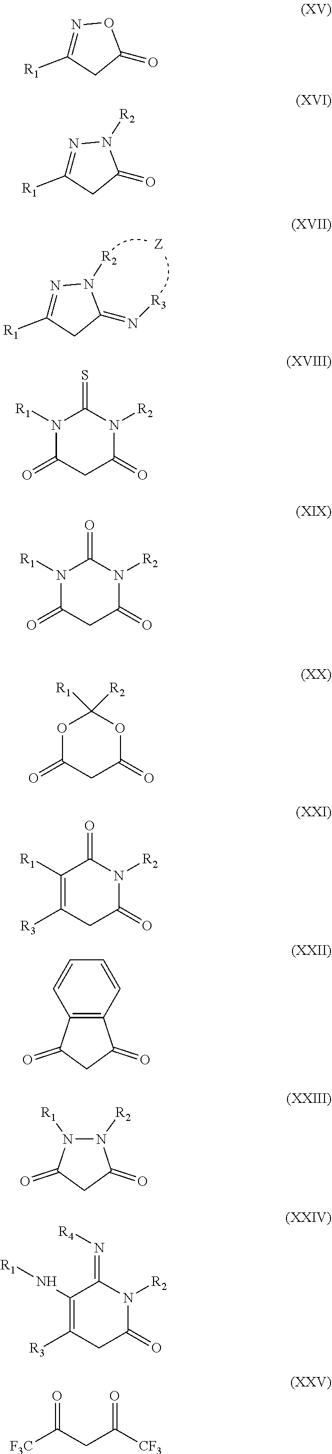

where R¹-R⁴ are independently one or more of alkyl, —C═O, COOH, COO(C₁-C₂₀ alkyl), COO(aryl), aryl, heteroaryl, C₁-C₂₀ alkoxy, C₁-C₂₀ aryloxy, O(alkyl), O(aryl), S(alkyl), S(aryl), C═N-aryl, N═N-alkyl, C═NH, C═S, —CN, —CH═CH₂, —C═(CN)₂, NO₂, and azide; wherein each alkyl and aryl group of R¹-R⁴ are independently one or more of alkyl, aryl, C₁-C₆ alkyl, heteroaryl, halogen, azide, hydroxyl, alkoxy, amino, N(C₁-C₂₀ alkyl), mono- or di-(C C₁-C₂₀) alkylamino, and halo(C₁-C₂₀) alkoxy; and where Z¹ includes one or more of C, N, O, S, and Se. While Z¹ is described as including one or more of C, N, O, S, and Se, these shall not be limiting as Z¹ may include any nonmetallic atom that completes the cyclic ring. For example, the acceptor group may be Meldrum's acid, barbituric acid, isoxazolone, pyrazolone, 1,5-benzodiazepine-2,4-dione, 1,3-indandione, 2-(3-oxo-indan-1-ylidene)-malononitrile, 1-alkyl-6-hydroxy-4-substituted-2-oxo-1,2-dihydropyridine-3-carbonitrile, and hexafluoroacetylacetone.

At step 103, at least the selected amine donor group and the selected acceptor group are contacted to form the negative photochromatic material. In other embodiments, additional materials may also be contacted to form the negative photochromatic material. In some embodiments, an oxygen-bearing heterocyclic compound (e.g., furan, furfural, etc.) may be included to form, for example, the polymethine chain bearing an oxygen heteroatom of the negative photochromatic material. In other embodiments, the acceptor group and the oxygen-bearing heterocyclic compound may be contacted to form an intermediate compound first and then the intermediate compound including at least the acceptor group and the oxygen-bearing heterocyclic compound may be contacted with the amine donor group to form the negative photochromatic material. In other words, in some embodiments, the acceptor group may refer to the intermediate compound that includes one or more of the chemical structures described above (e.g., XV to XXVI) and an oxygen-bearing heterocyclic compound.

One or more of the selected amine donor group and the selected acceptor group may tune at least an absorption range of the negative photochromatic material. The selection of either the amine donor group or acceptor group may tune the absorption wavelength of the negative photochromatic material to between a range of about 400 nm to about 800 nm (e.g., about 530 nm to about 700 nm). For example, the absorption wavelength may be tuned via selection of R¹, R², or both R¹ and R² of an amine donor group. In many embodiments, the negative photochromatic material is unreactive towards ultraviolet radiation. The amine donor group and/or the acceptor group may be selected to tune and/or modify properties of the negative photochromatic material. In one embodiment, the amine donor group and the acceptor may be selected to control and/or modify a basicity and/or conjugation of the negative photochromatic material to be formed. As the negative photochromatic material becomes more basic and/or more conjugated, the negative photochromatic material absorbs radiation at increasing wavelengths. For example, an increase in conjugation from N-methyl aniline to tetrahydroquinoline to indoline results in successive 15 nm shifts to the red. As another example, p-methoxyindole results in a redshift of about 13 nm relative to H-indole; and p-diheptylaminoindoline results in a redshift of about 45 nm relative to indoline. Increasing the planarity of the amine donor group relative to the conjugated system also leads to a red shift. In another embodiment, the absorption wavelength of the negative photochromatic material may be tuned by altering the group para or ortho to the nitrogen of the amine donor group and/or by using cyclic secondary anilines (e.g., tetrahydroquinoline or indoline).

In another embodiment, the amine donor group may be selected to tune, either to further tune or tune in the alternative, and/or control the photoswitchability of the negative photochromatic material in different media (e.g., any of the media described herein). In many embodiments, complete photoswitching or nearly complete photoswitching, from a colored form to a colorless form, occurs in any of the media described herein. In many embodiments, the most important feature are where R¹ and R² are independently one or more of alkyl, COOH, COO(C₁-C₂₀ alkyl), COO(aryl), aryl, heteroaryl, C₁-C₂₀ alkoxy, C₁-C₂₀ aryloxy, O(alkyl), O(aryl), S(alkyl) and S(aryl); wherein each alkyl and aryl group of R¹ and R² are independently one or more of alkyl, aryl, C₁-C₆ alkyl, heteroaryl, halogen, azide, hydroxyl, alkoxy, amino, N(C₁-C₂₀ alkyl), mono- or di-(C C₁-C₂₀) alkylamino, and halo(C₁-C₂₀) alkoxy. In some embodiments, R¹, R², or both R¹ and R² of an amine donor group may be selected to enable switching in solution or solid phase. For example, R¹, R², or both R¹ and R² of the amine donor group may be selected to enable switching in one or more of toluene, 1,4-dioxane, xylenes, anisole, dimethyl sulfoxide (DMSO), tetrahydrofuran (THF), water, methanol, ethanol, acetonitrile, chlorobenzene, N-methylpyrrolidone, dichlorobezene, trichlorobenzene, methylene chloride, acetone, benzene, cyclohexane, hexanes, ethyl acetate, diethyl either, 1,2-dichloroethane, and chloroform. In other embodiments, R¹, R², or both R¹ and R² of the amine donor group may be selected to enable switching in a polymer matrix. For example, R¹, R², or both R¹ and R² of the amine donor group may be selected to enable switching in polymer compositions including one or more of (methy)acrylate, (meth)acrylamide, (meth)acrylonitrile, styrene, acrylonitrile, vinyl acetate, vinylcarbazole, vinylpyridine, vinyl ether, vinyl chloride, and siloxane monomers. In other embodiments, R¹, R², or both R¹ and R² of an amine donor group may be selected to enable switching in a solid matrix, wherein the solid matrix includes one or more of paper, nylon, cotton, fibers, and membranes.

The amine donor group and/or acceptor group may also be selected to tune, either to further tune or tune in the alternative, and/or control a temperature dependence of the thermal reversion of the negative photochromatic material. In another embodiment, the amine donor group and/or acceptor group may be selected to tune a temperature dependence of the thermal reversion. The R group on one or more donor or acceptor groups can be used to modify the thermal activation energy required to convert the colorless material to the colored material. In some embodiments, R¹, R², or both R¹ and R² of the amine donor group may be selected to tune a temperature dependence of the thermal reversion. In other embodiments, X, Y, and R⁵ of the acceptor group may be selected to tune a temperature dependence of the thermal reversion. In another embodiment, the polymer matrix may be selected to tune a temperature dependence of the thermal reversion.

### EXAMPLE 1

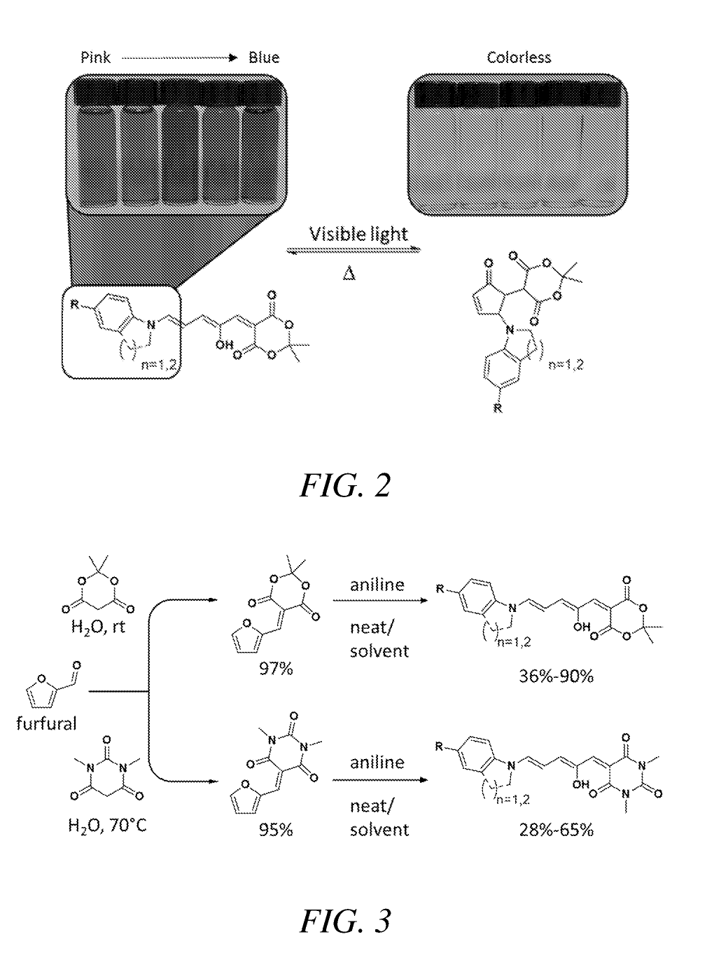

This example describes, among other things, the use of secondary anilines as donors to produce DASA molecules with highly tunable absorption wavelengths. (FIG. 2). The wavelength may be altered by, for example, altering the group para to the nitrogen as well as using cyclic secondary anilines such as tetrahydroquinoline or indoline. Complete photoswitching from a colored to a colorless form was observed in a range of solvents and in a polymer matrix. Embodiments provide a family of photochromic material which can be prepared economically and easily. Further provided is a family of photochromic material which provides the ability to control the conversion between forms of the photochromic material with wavelengths that range between about 400 and about 800 nm.

A class of highly tunable visible and near-infrared donor-acceptor Stenhouse adduct (DASA) photoswitches were efficiently synthesized and characterized to reveal unique structure property relationships. Variations in color was the starkest property that could be altered, providing a desirable absorption range spanning from 400 to 800 nm. The synthesis is accomplished in 2 to 4 steps from commercially available starting materials with minimal purification. The utility of highly tunable photoswitches is demonstrated through selective switching of one of two photoswitches in a mixture. The mixture can be in solution or in a solid matrix.

Examples of acceptors include:

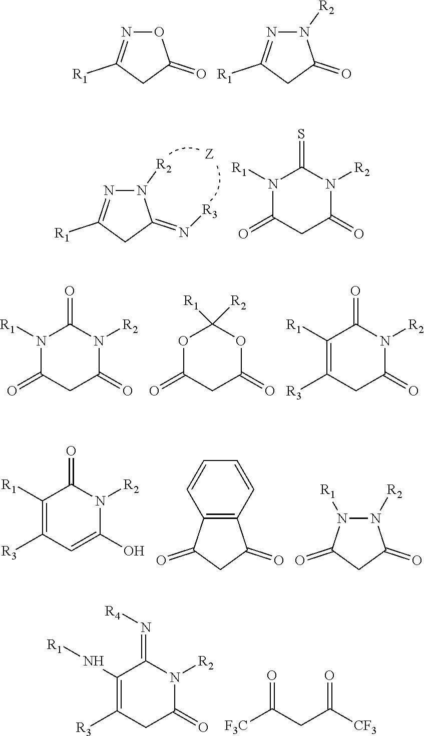

where each of R¹, R², R³, and R⁴ are independently one or more of alkyl, —C═O, COOH, COO(C₁-C₂₀ alkyl), COO(aryl), aryl, heteroaryl, C₁-C₂₀ alkoxy, C₁-C₂₀ aryloxy, O(alkyl), O(aryl), S(alkyl), S(aryl), C═N-aryl, N═N-alkyl, C═NH, C═S, —CN, —CH═CH₂, —C═(CN)₂, NO₂, and azide; and where Z includes one or more of C, N, O, S, and Se. In some embodiments, each alkyl or aryl group of R¹-R⁴ may be optionally substituted with one or more groups that are independently one or more of alkyl, aryl, C₁-C₆ alkyl, heteroaryl, halogen, azide, hydroxyl, alkoxy, amino, N(C₁-C₂₀ alkyl), mono- or di-(C C₁-C₂₀) alkylamino, and halo(C₁-C₂₀) alkoxy. In addition, while Z¹ is described as including one or more of C, N, O, S, and Se, these shall not be limiting as Z¹ may include any nonmetallic atom that completes the cyclic ring.

In general, the disclosed methods provide improvements in the preparation of the negative photochromic material. A fundamental advantage of this photochromic material is that UV light is not required. Further, the photochromic material of this invention has an absorption maximum that can be tuned and is in the range of wavelengths 400 to 800 nm. It is understood that the R¹ and R² on the amine group can be modified to further shift the wavelength of the photochromic material to either longer or shorter wavelengths as known in the art.

The photochromic material described herein can be operated in any suitable solvent known in the art, depending on the desired need, for example toluene, 1,4-dioxane, xylenes, anisole, dimethyl sulfoxide (DMSO), tetrahydrofuran (THF), water, methanol, ethanol, acetonitrile, chlorobenzene, N-methylpyrrolidone, dichlorobezene, trichlorobenzene, methylene chloride, acetone, benzene, cyclohexane, hexanes, ethyl acetate, diethyl either, 1,2-dichloroethane, chloroform and the like. In addition, in some embodiments of the invention, the photochromic material may also be incorporated and operate in polymer systems.

Examples are included in Table 1 which is not intended to limit the current embodiments in any way.

TABLE 1
EXAMPLARY DYES

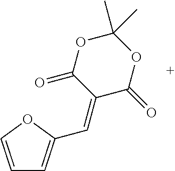 [SMILES: CC1(C)OC(=O)C(=Cc2ccco2)C(=O)O1]

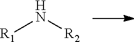

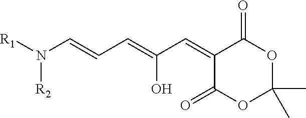

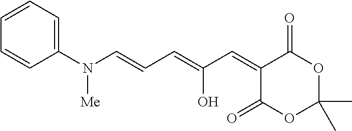 [SMILES: CN(/C=C/C=C(\O)C=C1C(=O)OC(C)(C)OC1=O)c1ccccc1]

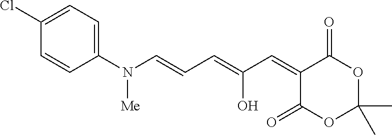 [SMILES: CN(/C=C/C=C(\O)C=C1C(=O)OC(C)(C)OC1=O)c1ccc(Cl)cc1]

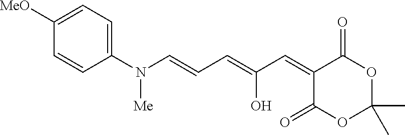 [SMILES: COc1ccc(N(C)/C=C/C=C(\O)C=C2C(=O)OC(C)(C)OC2=O)cc1]

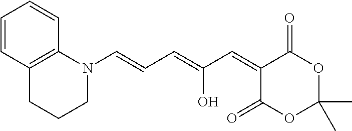 [SMILES: CC1(C)OC(=O)C(=C/C(O)=C/C=C/N2CCCc3ccccc32)C(=O)O1]

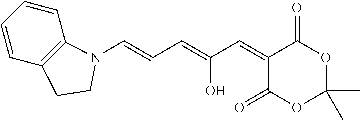 [SMILES: CC1(C)OC(=O)C(=C/C(O)=C/C=C/N2CCc3ccccc32)C(=O)O1]

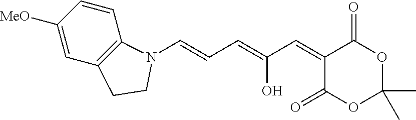 [SMILES: COc1ccc2c(c1)CCN2/C=C/C=C(\O)C=C1C(=O)OC(C)(C)OC1=O]

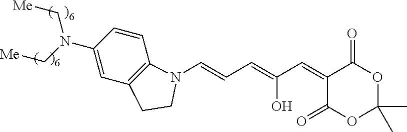

TABLE 2
EXAMPLARY DYES

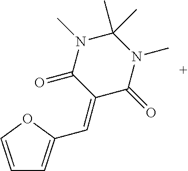 [SMILES: CN1C(=O)C(=Cc2ccco2)C(=O)N(C)C1(C)C]

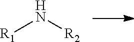

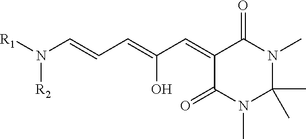

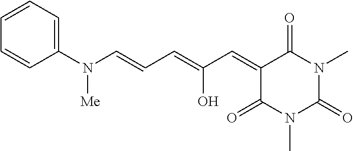 [SMILES: CN1C(=O)C(=C/C(O)=C/C=C/N(C)c2ccccc2)C(=O)N(C)C1=O]

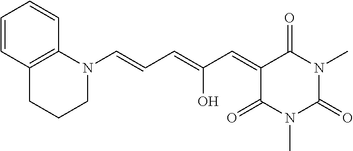 [SMILES: CN1C(=O)C(=C/C(O)=C/C=C/N2CCCc3ccccc32)C(=O)N(C)C1=O]

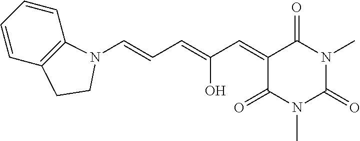 [SMILES: CN1C(=O)C(=C/C(O)=C/C=C/N2CCc3ccccc32)C(=O)N(C)C1=O]

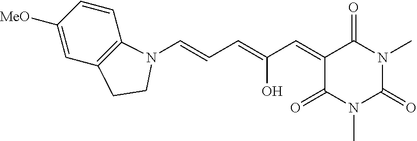 [SMILES: COc1ccc2c(c1)CCN2/C=C/C=C(\O)C=C1C(=O)N(C)C(=O)N(C)C1=O]

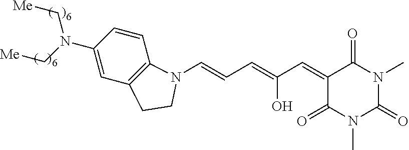

The photochromic material represented by formula (I) can be synthesized according to the process described in J. Am. Chem. Soc. 2016, 138, 13960 or according to the process as given in the following synthesis examples.

### SYNTHESIS EXAMPLE 2

### Synthesis of Photochromic Material with Meldrum's Acid Acceptor

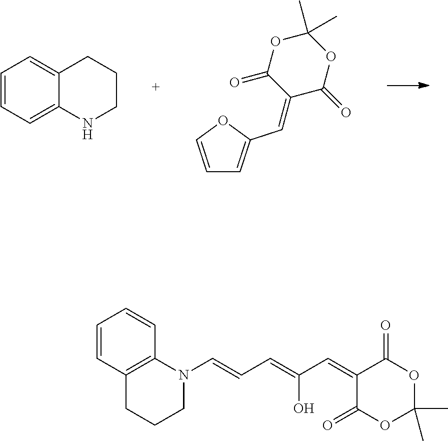 [SMILES: c1ccc2c(c1)CCCN2 ; CC1(C)OC(=O)C(=Cc2ccco2)C(=O)O1 ; CC1(C)OC(=O)C(=C/C(O)=C/C=C/N2CCCc3ccccc32)C(=O)O1]

222.22 mg (1 mmol) of Meldrum's acid furan adduct and 250 μL (2 mmol) tetrahydroquinoline were combined and allowed to stir for 4 hours. 2 mL of THF was added to the resulting residue and the residue was broken up and sonicated. This was then filtered to yield 184 mg (51% yield).

### SYNTHESIS EXAMPLE 3

### Synthesis of Photochromic Material with Barbituric Acid Acceptor

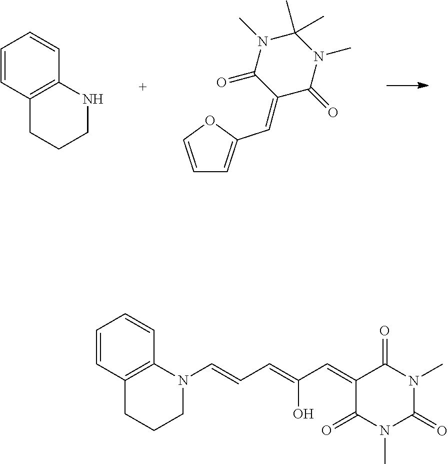 [SMILES: c1ccc2c(c1)CCCN2 ; CN1C(=O)C(=Cc2ccco2)C(=O)N(C)C1(C)C ; CN1C(=O)C(=C/C(O)=C/C=C/N2CCCc3ccccc32)C(=O)N(C)C1=O]

234.21 mg (1 mmol) Barbituric acid furan adduct and 103 μL (1 mmol) of diethylamine were stirred in 4 mL THF. After 45 minutes, the precipitate was filtered and washed with diethyl ether. The product was then allowed to dry.

### EXAMPLE 4

### Synthesis and Testing of Photochromic Material with either Barbituric Acid Acceptor or Meldrum's Acid Acceptor

Synthesis of aniline based DASAs is a two-step process from commercially available starting materials (FIG. 3). First, an activated furan-carbon acid is synthesized. Furfural, an inexpensive material made from non-edible biomass, is condensed with either Meldrum's or barbituric acid to create an activated furan adduct in high yield. This synthesis is performed in water either at room temperature or 70° C. to yield a yellow precipitate. The product can then be isolated through filtration and purified by washing with water.

Next, this activated furan adduct is combined with a secondary aniline. Non-basic anilines such as N-methyl can be reacted neat using 3-5 equivalents of the aniline to the furan adduct. The product is isolated as a crystalline solid following trituration with diethyl ether or hexanes. More electron rich anilines, such as p-OMe-N-methylaniline or indoline can be run with 1 equivalent of aniline in a solvent such as THF or DCM. Again, purification by trituration with diethyl ether or THF led to the isolation of a pure crystalline product.

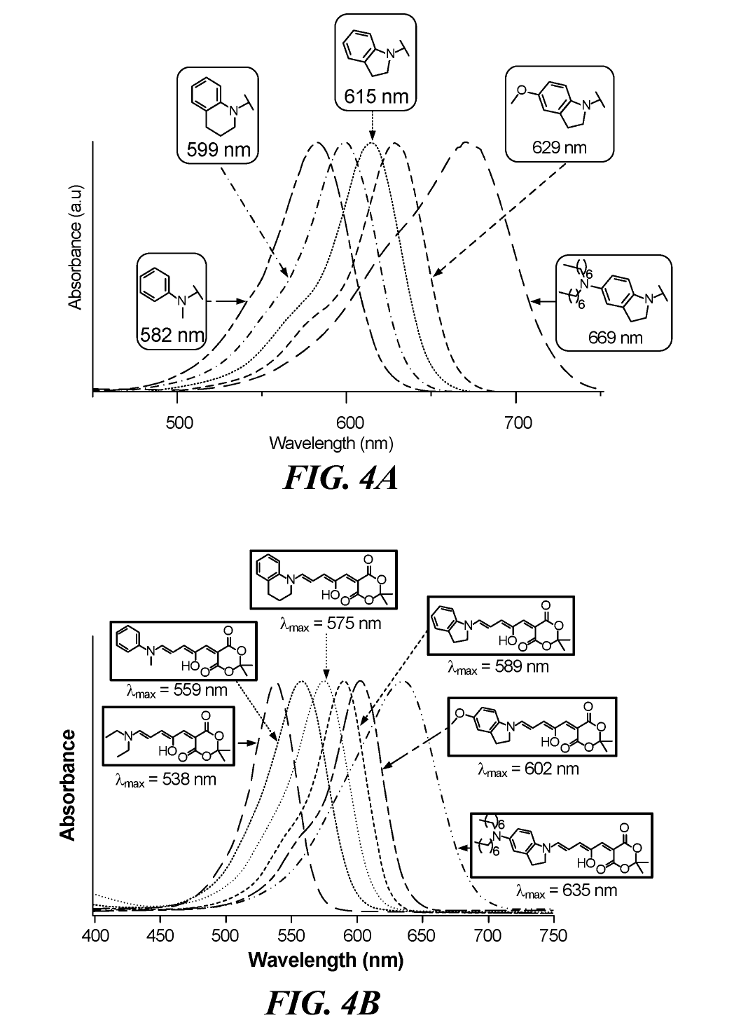

With optimized synthesis conditions in hand, a small library of DASAs was synthesized using a variety of aniline donors and their absorption wavelengths characterized. The absorption spectra of these compounds is highlighted in FIGS. 4A-B. Starting from the di-alkylamine (abs. 570 nm) donors, a steady increase in wavelengths is seen through the aniline based DASAs as they become more basic and more conjugated. The increase in conjugation from N-methyl aniline to tetrahydroquinoline to indoline results in successive 15 nm shifts to the red. Electron donating substituted indolines display a further redshift in absorbance. Compared to H-indole, p-methoxyindole gives a redshift of 13 nm. Finally, the highly donating p-diheptylaminoindoline gives a 45 nm redshift compared to indoline. A nearly 100 nm shift from the original dialkylamine DASA was achieved simply by changing the aniline used. This wavelength control expands the potential use of this photoswitch in a variety of applications as well as open the door for selective switching of a single DASA in a mixture of photoswitches.

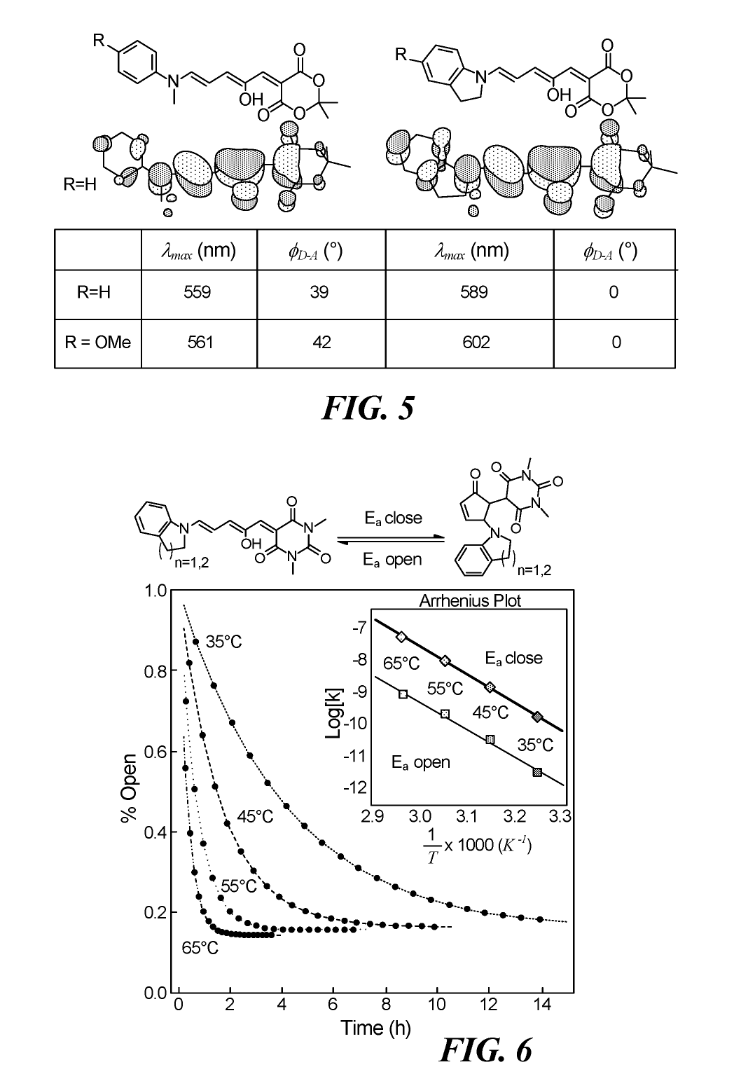

Next, a series of DFT measurements were conducted on the molecules synthesized in order to understand the origin of their spectroscopic properties. FIG. 5 shows the computationally derived geometry and HOMO orbitals for the N-methylaniline and indolines donor DASAs as well as the dihedral angles of the phenyl rings compared to the triene backbone. The dihedral angle of the phenyl ring in N-methyl aniline is 39° out of the plane of conjugation. In contrast, the indoline moity lies completely in plane with the rest of the molecule and has a 0° twist out of plane. The effect of the phenyl ring being in or out of plane can clearly be seen in the contributions of the phenyl ring to the HOMO orbitals. While the phenyl ring in N-methyl aniline has some contribution to the HOMO, the contribution of the in-plane indole is much greater.

The effect of the change in dihedral angle becomes apparent when comparing the wavelengths of these two aniline DASAs. The additional conjugation between the aromatic ring and the triene gives a 30 nm redshift between the aniline and indoline derivatives. Even more striking is the effect of a paramethoxy donating group on both the aniline and indolines derivatives. Since the N-methylaniline is out of plane, the donating group has little impact on the wavelength and only causes a 2 nm bathochromic shift. However, in the indoline case, the addition of a paramethoxy group causes a 13 nm redshift. Conjugation of the aryl ring in the aniline donor is critical for increasing the wavelength.

The fundamental kinetic properties of these photochromic materials was investigated. The opening and closing rates of the aniline DASAs as well as their opening and closing activations were studied by NMR spectroscopy. Samples of N-methylaniline, tetrahydroquinoline, and indolines based DASAs were dissolved in dichlorobenzene and allowed to reach their equilibrium open-closed value. The rate of closing was then modeled. Using variable temperature NMR, the rates could be monitored at various temperatures. By plotting the rate constants vs 1/T the Arrhenius expression was used to give activation energies for both the opening and closing of the DASA (FIG. 6).

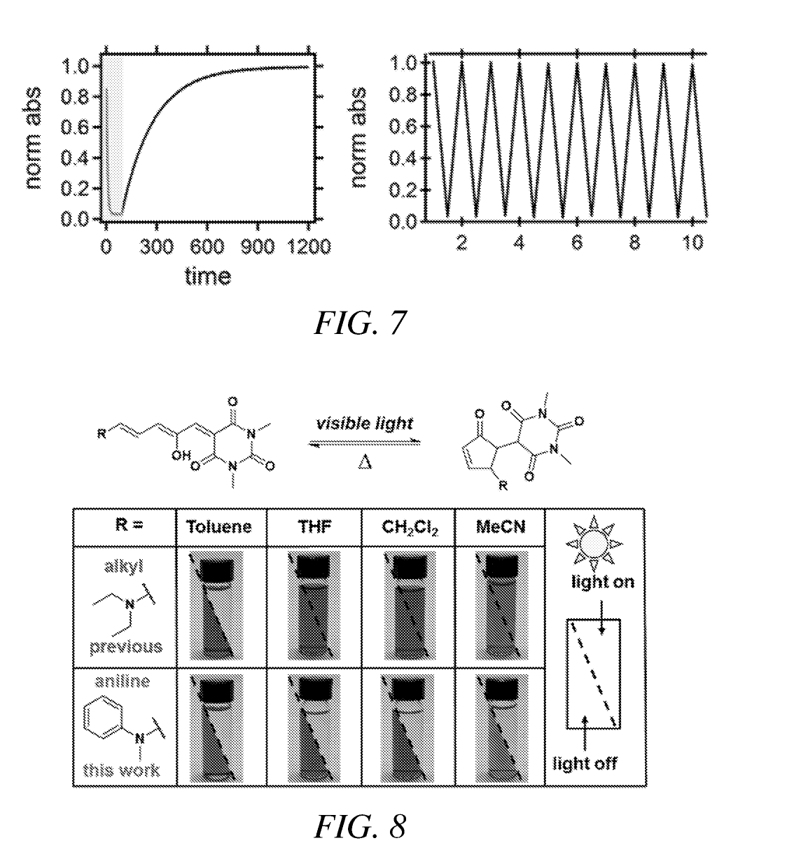

Reversibility and stability are important in photochromic systems. Spiropyrans can fatigue quickly upon repeated exposure with UV light. For spiropyrans, about 50% degradation may be observed after 13 cycles. An advantage of a visible light chromophore is that it uses longer wavelength, lower energy light to elicit photoisomerization, which often results in increased stability. To test the robustness of the aniline based DASA system, extensive cycling tests were performed with the para-methoxy indoline barbituric acid derivative (FIG. 7). Similar to the previously reported dialkyl DASAs, almost no degradation was observed after 10 cycles. After 100 cycles, the absorbance recovered 80% percent of its original value. After 200 cycles, 60% recover was observed. This demonstrates the significant stability of Stenhouse adducts, and highlights the utility of visible light absorbing photoswitches.

Photoswitching of previously reported dialkyl DASAs was limited to non-polar solvents such as toluene, xylenes, and dioxane. In more polar solvents like THF or dichloromethane, no photoisomerization could be observed even with intense lights sources. Trends from the dialkyl DASAs carry though into the aniline based DASAs. In general, switching to the closed of the DASA occurs more readily in more non-polar solvents such as hexanes or toluene, not considering solubility issues. More polar solvents like THF or methylene chloride strongly favor the open form. However, aniline based DASAs undergo photoisomerization in a variety of solvents due to their decreased basicity when compared to alkyl DASAs. FIG. 8 highlights the ability of the N-methyl aniline-barbituric acid DASA to undergo full photoisomerization to the cyclized form in a variety of solvents, while dialkyl derivative cyclizes only in toluene. The ability of aniline based DASAs to photoswitch in multiple solvents expands the utility of the DASA system and is important for enabling reversible switching in polymer matrices.

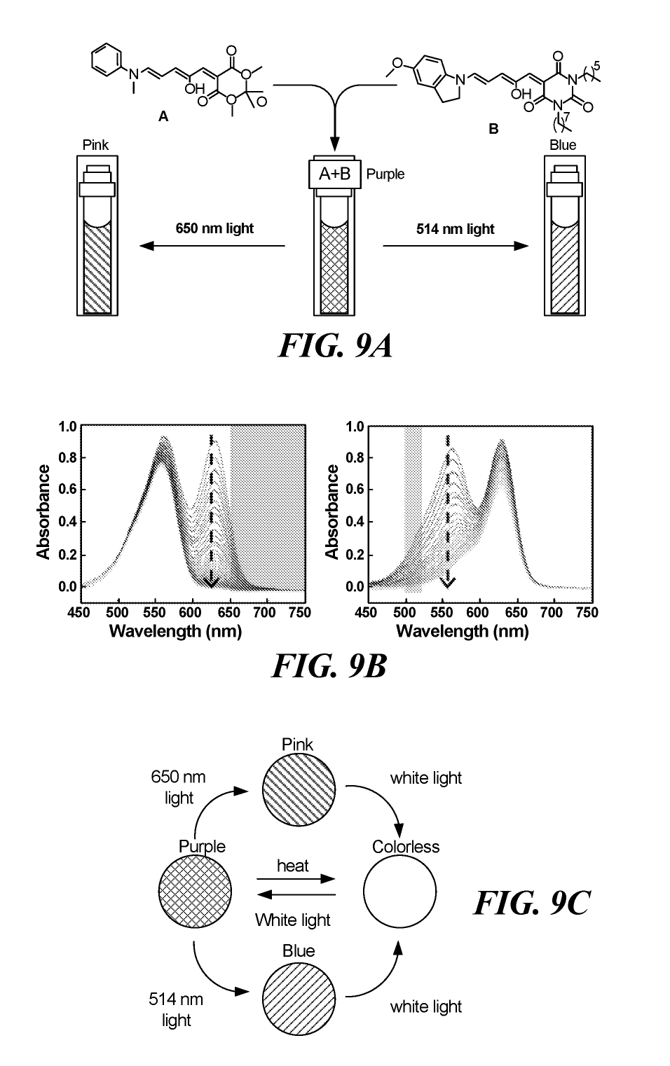

The utility of having a range of absorption spectra was demonstrated by selectively switching one DASA in a system comprised of two DASAs. DASAs with a large gap in their absorption may allow selective photoswitching in response to different wavelengths of light. N-methylaniline on Meldrum's acid A and p-methoxyindoline on alkylated barbituric acid B were chosen, which have a maximum absorption at 560 nm and 623 nm, respectively (FIG. 9A). Next, solution containing both A (10 μM) and B (6 μM) in toluene was prepared and the absorbance of the DASA mixture monitored with increasing irradiation time. First, a long-pass LED light which only passes wavelengths above 650 nm was used and irradiated for 30 minutes (FIG. 9B). The absorption peak at 623 nm corresponding to B selectively decreased with time, resulting in the color change of the solution from purple to pink. Of note, the slight decrease at 560 nm is irrelevant to photoswitching of A, evidenced by two control experiments of single DASAs. The filtered light with 514 nm band-pass was then used, which has a great overlap with A and a slight overlap with B. Irradiation for 10 minutes led to a rapid decrease in absorption at 560 nm corresponding to A and only a slight decrease at 623 nm (FIG. 9B). As a result, the purple color of the DASA mixture turned blue.

Selective switching in solid state was further investigated. In a solution of 100 mg/mL PMMA (350 kDa) in dichloromethane, A+B (0.025 wt.% of A and 0.015 wt.% of B) were added and drop-casted onto a glass slide (FIG. 9C). Irradiation with 650 nm LED for 10 minutes, which resulted the color change of A+B sample from purple to pink due to selective photoswitching of B. On the other hand, irradiating with 450 nm LED for 3 minutes led A+B sample to change from purple to blue from selective photoswitching of A. Using a broad spectrum light, all samples were switched to their colorless and photoswitched forms, which reverted back to its original colored state by heat. This demonstrates the ability to effectively selectively switch one DASA in the presence of the other, as well as the reversible switching of DASA in a polymer matrix.

In summary, embodiments describe a highly tunable photoswitching molecular platform. Through careful choice of the donor moiety, the wavelength and solvent switching properties of aryl DASAs can be precisely controlled. The ability to modify wavelength on demand and selective switch different DASAs allows for selective switching of one DASA in the presence of another. These properties will enable the use of this photochromic system in a variety of applications.

Other embodiments of the present disclosure are possible. Although the description above contains much specificity, these should not be construed as limiting the scope of the disclosure, but as merely providing illustrations of some of the presently preferred embodiments of this disclosure. It is also contemplated that various combinations or sub-combinations of the specific features and aspects of the embodiments may be made and still fall within the scope of this disclosure. It should be understood that various features and aspects of the disclosed embodiments can be combined with or substituted for one another in order to form various embodiments. Thus, it is intended that the scope of at least some of the present disclosure should not be limited by the particular disclosed embodiments described above.

Thus the scope of this disclosure should be determined by the appended claims and their legal equivalents. Therefore, it will be appreciated that the scope of the present disclosure fully encompasses other embodiments which may become obvious to those skilled in the art, and that the scope of the present disclosure is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims.

The foregoing description of various preferred embodiments of the disclosure have been presented for purposes of illustration and description. It is not intended to be exhaustive or to limit the disclosure to the precise embodiments, and obviously many modifications and variations are possible in light of the above teaching. The example embodiments, as described above, were chosen and described in order to best explain the principles of the disclosure and its practical application to thereby enable others skilled in the art to best utilize the disclosure in various embodiments and with various modifications as are suited to the particular use contemplated. It is intended that the scope of the disclosure be defined by the claims appended hereto.

1. A negative photochromic material, comprising:
a negative photochromatic material characterized by the formula: 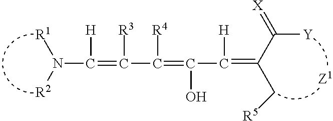
where R¹ and R² are independently one or more of alkyl, COOH, COO(C₁-C₂₀ alkyl), COO(aryl), aryl, heteroaryl, C₁-C₂₀ alkoxy, C₁-C₂₀ aryloxy, O(alkyl), O(aryl), S(alkyl) and S(aryl); wherein each alkyl and aryl group of R¹ and R² are independently one or more of alkyl, aryl, C₁-C₆ alkyl, heteroaryl, halogen, azide, hydroxyl, alkoxy, amino, N(C₁-C₂₀ alkyl), mono- or di-(C C₁-C₂₀) alkylamino, and halo(C₁-C₂₀) alkoxy;
where R³ and R⁴ are independently one or more of hydrogen, alkyl groups, aryl groups, —S(aryl), —S(heteroaryl), —S(alkyl), —O(alkyl), —O(aryl), —O(heteroaryl), azide, and halogen;
where R⁵ is independently one or more of —C═O, C═N-aryl, N═N-alkyl, C═NH, C═S, —CN, —CH═CH₂, CF₃, halo(C₁-C₂₀), alkyl, —COOH, —COO(C₁-C₂₀ alkyl), —COO(aryl), aryl, and heteroaryl;
where X is independently O, N-aryl, N-alkyl, N—H, —(CN)₂, and S;
where Y is independently one or more of O, N-alkyl, N-aryl, N-heteroaryl, S, alkyl, and aryl; and
where Z¹ includes one or more of C, N, O, S, and Se.

2. The material of claim 1, wherein the negative photochromatic material is characterized by one or more of the following chemical structures:

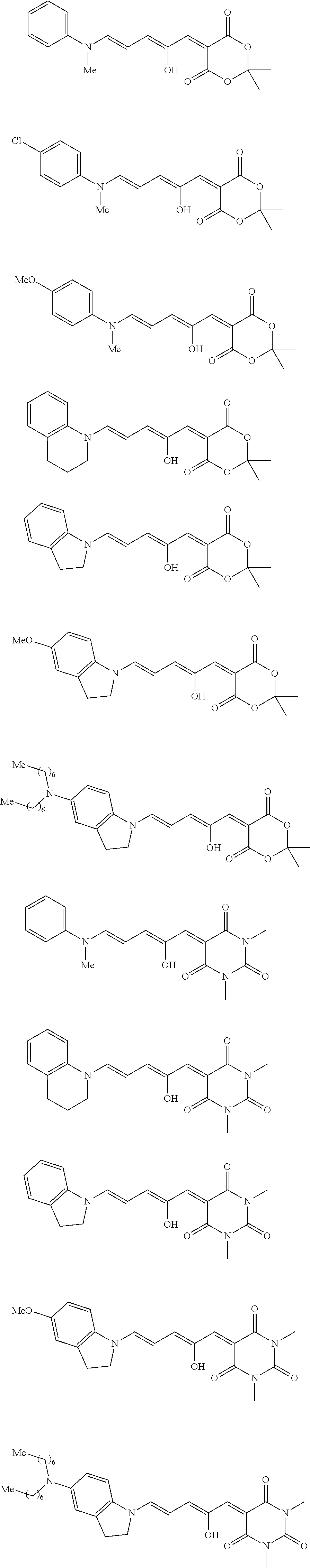

3-9. (canceled)

10. The material of claim 1, wherein R¹, R², or both R¹ and R² of an amine donor group is selected to enable switching in solution or solid phase.

11. The material of claim 1, wherein R¹, R², or both R¹ and R² of an amine donor group is selected to enable switching in one or more of toluene, 1,4-dioxane, xylenes, anisole, dimethyl sulfoxide (DMSO), tetrahydrofuran (THF), water, methanol, ethanol, acetonitrile, chlorobenzene, N-methylpyrrolidone, dichlorobezene, trichlorobenzene, methylene chloride, acetone, benzene, cyclohexane, hexanes, ethyl acetate, diethyl either, 1,2-dichloroethane, and chloroform.

12. The material of claim 1, wherein R¹, R², or both R¹ and R² of an amine donor group is selected to enable switching in a polymer matrix.

13. The material of claim 12, wherein the amine donor group is selected to enable switching in polymer compositions including one or more of (methy)acrylate, (meth)acrylamide, (meth)acrylonitrile, styrene, acrylonitrile, vinyl acetate, vinylcarbazole, vinylpyridine, vinyl ether, vinyl chloride, and siloxane monomers.

14. The material of claim 1, wherein R¹, R², or both R¹ and R² of an amine donor group is selected to enable switching in a solid matrix.

15. (canceled)

16. The material of claim 1, wherein the negative photochromatic material converts from a thermodynamically stable state to a photostationary state upon contact with electromagnetic radiation.

17. (canceled)

18. A method of tuning a negative photochromatic material, comprising:
selecting an amine donor group characterized by the formula: 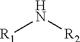
where R¹ and R² are independently one or more of alkyl, COOH, COO(C₁-C₂₀ alkyl), COO(aryl), aryl, heteroaryl, C₁-C₂₀ alkoxy, C₁-C₂₀ aryloxy, O(alkyl), O(aryl), S(alkyl), S(aryl); wherein each alkyl and aryl group of R¹ and R² are independently one or more of alkyl, aryl, C₁-C₆ alkyl, heteroaryl, halogen, azide, hydroxyl, alkoxy, amino, N(C₁-C₂₀ alkyl), mono- or di-(C C₁-C₂₀) alkylamino, and halo(C₁-C₂₀) alkoxy;
selecting an acceptor group; and
contacting at least the selected amine donor group and the selected acceptor group to form a negative photochromatic material;
wherein one or more of the selected amine donor group and the selected acceptor group tune at least an absorption range of the negative photochromatic material.

19. The method of claim 18, wherein the amine donor group is a secondary aromatic amine.

20. The method of claim 18, wherein the amine donor group is one or more of N-methylaniline, N-ethylaniline, N-propylaniline, N-butylaniline, N-pentylaniline, N-hexylaniline tetrahydroquinoline, indoline, 5-methoxyindoline, 5-ethoxyindoline, 5-methoxy-2-methylindoline, 5-ethoxy-2-methylindoline, N,N-dimethylindolin-5-amine, N,N-diethylindolin-5-amine, N,N-dipropylindolin-5-amine, N,N-dibutylindolin-5-amine, N,N-dipentylindolin-5-amine, N,N-dihexylindolin-5-amine, N,N-dimethy-2-methylindolin-5-amine, N,N-diethyl-2-methylindolin-5-amine, N,N-dipropy-2-methylindolin-5-amine, N,N-dibutyl-2-methylindolin-5-amine, N,N-dipenty-2-methylindolin-5-amine, N,N-dihexyl-2-methylindolin-5-amine.

21. The method of claim 18, wherein the acceptor group is one or more of the following: 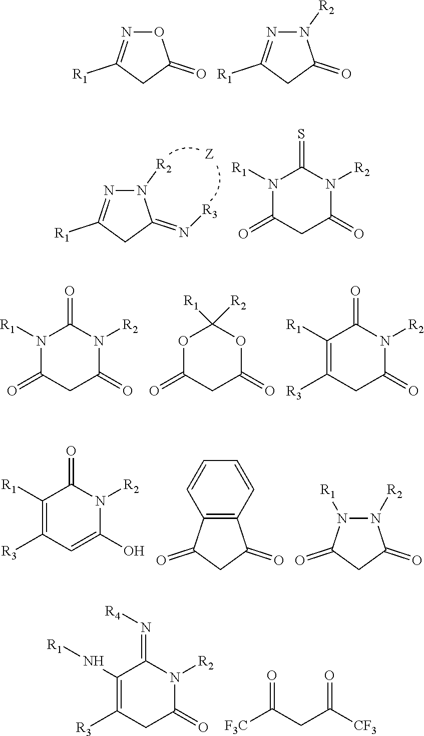
where R¹-R⁴ are independently one or more of alkyl, —C═O, COOH, COO(C₁-C₂₀ alkyl), COO(aryl), aryl, heteroaryl, C₁-C₂₀ alkoxy, C₁-C₂₀ aryloxy, O(alkyl), O(aryl), S(alkyl), S(aryl), C═N-aryl, N═N-alkyl, C═NH, C═S, —CN, —CH═CH₂, —C═(CN)₂, NO₂, and azide;
wherein each alkyl and aryl group of R¹-R⁴ are independently one or more of alkyl, aryl, C₁-C₆ alkyl, heteroaryl, halogen, azide, hydroxyl, alkoxy, amino, N(C₁-C₂₀ alkyl), mono- or di-(C C₁-C₂₀) alkylamino, and halo(C₁-C₂₀) alkoxy;

22. The method of claim 18, wherein the acceptor group is one or more of Meldrum's acid, barbituric acid, isoxazolone, pyrazolone, 1,5-benzodiazepine-2,4-dione, 1,3-indandione, 2-(3-oxo-indan-1-ylidene)-malononitrile, 1-alkyl-6-hydroxy-4-substituted-2-oxo-1,2-dihydropyridine-3-carbonitrile, and hexafluoroacetylacetone.

23. (canceled)

24. The method of claim 18, wherein the absorption range is tuned to a range from about 400 nm to about 800 nm.

25. (canceled)

26. The material of claim 18, wherein the amine donor group is selected to further tune a temperature dependence of the thermal reversion.

27. The method of claim 18, wherein the acceptor group is selected to further tune a temperature dependence of the thermal reversion.

28. The method of claim 18, wherein the polymer glass transition temperature (T_{g}) is selected to further tune a temperature dependence of the thermal reversion.

29. The method of claim 18, wherein the negative photochromatic material converts from a thermodynamically stable state to a photostationary state upon contact with electromagnetic radiation.

30-31. (canceled)

32. (canceled)

33. The method of claim 18, wherein the amine donor group is selected to enable photoswitching in a polymer matrix.

34. (canceled)

35. The method of claim 18, wherein the amine donor group is selected to enable photoswitching in a solid matrix.

36-38. (canceled)
